(19) United States
(12) Patent Application Publication
(10) Pub. No.: US 2010/0227356 A1
(43) Pub. Date: Sep. 9, 2010

(54) METHODS FOR ASSESSING EFFECTS ON SKELETAL GROWTH

(76) Inventor: Rao Papineni, Branford, CT (US)

Correspondence Address: Carestream Health, Inc.;ATTN: Patent Legal Staff, 150 Verona Street, Rochester, NY, 14608, US

(21) Appl. No.: 12/553,146

(22) Filed: Sep. 3, 2009

Related U.S. Application Data
(60) Provisional application No. 61/094,135, filed on Sep. 4, 2008.

(51) Int. Cl. C12Q 1/02

(52) U.S. Cl. 435/29

(57) ABSTRACT

A method for assessing effects on growth of skeletal members comprises preparing a culture comprising a skeletal member and a substance whose effect on growth of the skeletal member is to be determined and capturing images of the skeletal member at selected time intervals without terminating growth. The skeletal member may be fully articulated and the substance whose effect on growth is to be determined may be labeled with a marker and tracked over time. The methods of the invention may be used to assess effects on growth through the positioning of the substance in the skeletal member and for drug discovery.

### CROSS-REFERENCE TO RELATED APPLICATION

This is a 111A application of Provisional Application U.S. Ser. No. 61/094,135, filed 4 Sep. 2008, entitled “SOFT X-RAY IMAGING OF IN-VITRO GROWTH OF NEONATAL ORGAN-BONE” by Rao Papineni.

### FIELD OF THE INVENTION

This invention relates generally to the field of methods for assessing the effects of various substances on skeletal growth and more particularly to assessing those effects by imaging fully articulated skeletal members at selected time points in vitro over their growth cycles.

### BACKGROUND OF THE INVENTION

In vitro models of in vivo systems are useful for studying normal system development, disease states, and the effect of drugs and/or toxins on such systems. In vitro models are useful because the environment of the system to be analyzed can be tightly controlled, thereby allowing for isolation of a single aspect for study.

The use of an organ culture provides an opportunity to more closely recreate in vivo conditions with respect to the cells that are part of said organ. Methods for maintaining organs, such as bone, in organ culture, are known. For example, the process of skeletal growth in fetal mouse bones has been monitored and measured by staining bones grown in organ culture. Suda discloses a method for studying the endochondral ossification process of skeletogenesis by correlating X-ray imaging with double staining using alcian blue and alizarin red. Suda, M. et al., Skeletal overgrowth in transgenic mice that overexpress brain natriuretic peptide, 95(5) PNAS 2337-2342 (1998). The dyes used by Suda destroy the cells and stop further growth and are thus only useful for end point studies.

In another example, fetal mouse metatarsals, grown in organ culture and exposed to the conditions experienced in outer space, were analyzed using light microscopy and electron microscopy combined with X-ray microanalysis. O. P. Berezovska, Features of fetal bone organ culture development under space flight conditions, 36 (2) Cytology and Genetics 60-67 (2002). As in Suda, the analysis once again relied upon fixing and staining the metatarsals, thereby interfering with the ability to track the dynamic processes of bone growth and mineral deposition.

While the prior art discloses methods for monitoring skeletal growth in vitro, the disclosed methods typically destroy the culture and terminate growth. It would be beneficial to have a system that would allow repeated measurements on the same culture in order to accurately follow changes in said culture over time.

### SUMMARY OF THE INVENTION

Methods of the present invention are used to assess effects on skeletal growth. By employing one or more of these methods, users can study bone development, mechanisms behind disease states of bone and the effect of drugs on bone development and disease.

In one embodiment, the method comprises imaging of a culture comprising a skeletal member and a substance whose effect on growth of the skeletal member is to be determined. The substance comprises a marker capable of at least one of fluorescence, radioactivity or radio-opacity and the culture enables growth of the skeletal member. The imaging may be multi-modal at two or more selected time points, t₀ and tₙ. The time point t₀ corresponds to a first time point at which the multi-modal imaging is conducted and tₙ corresponds to each successive time point thereafter. Based on the imaging, the positioning of the substance whose effect on growth is to be determined is identified at the selected time points t₀ and tₙ. In this way, one can assess whether the positioning of the substance effects growth of the skeletal member.

In another embodiment, the method comprises preparing a culture comprising a skeletal member and at least one cell type that effects growth of the skeletal member and tracking the positioning of cells during growth. The culture enables growth of the skeletal member and the at least one cell type comprises a marker capable of exhibiting at least one of fluorescence, radioactivity or radio-opacity. After the at least one cell type divides into a plurality of cells, images of the skeletal member at two or more selected time points are taken. The two or more selected time points are represented by t₀ and tₙ, where t₀ corresponds to a first time point at which the imaging is conducted and tₙ corresponds to each successive time point thereafter. The imaging does not terminate growth of the skeletal member. To assess how cell division effects growth of the skeletal member, images of the plurality of cells at the selected time points t₀ and tₙ are analyzed.

In yet another embodiment, the method comprises preparing two or more cultures comprising fully articulated skeletal members to assess the effects of drug candidates on skeletal growth. Both cultures enable growth of the skeletal member, with the first culture serving as a control and the second culture containing the drug candidate. Images of the skeletal members from each of the first and second cultures at two or more selected time points, t₀ and tₙ, are captured. The time point t₀ corresponds to a first time point at which the imaging is conducted and tₙ corresponds to each successive time point thereafter. The method further comprises assessing whether a drug candidate effects growth of the skeletal member by comparing the images captured at the selected time intervals t₀ and tₙ for each of the first and second cultures against each other.

Methods of the present invention can incorporate various additional features. Images captured at the two or more time points can be compared, by subtraction or superimposition of any of the two or more images, for example. The time points for comparison of the images and positioning of the substance whose effect on growth of the skeletal member is to be determined may be the same or different. The at least one cell type may comprise a feeder cell or progenitor cell or provide a growth factor. The number of divided cells and their positioning may also be analyzed. The skeletal member may be fully articulated or non-articulated. The imaging may be conducted by X-ray or fluorescent microscopy or both and includes multi-modal imaging. When the methods are used for drug discovery, a disease state may be introduced into the skeletal member to assess whether drug candidates treat the disease state. The skeletal member may also be injured (e.g., wounded or broken) to assess whether drug candidates treat injuries.

### BRIEF DESCRIPTION OF THE DRAWINGS

FIG. 1 is a schematic diagram of a multi-modal imaging system suitable for use in accordance with the present invention.

FIG. 2A illustrates two time-spaced X-ray images of a hind limb of a neonatal mouse grown in organ culture.

FIG. 2B is a schematic illustration showing changes in bone density and the distances between each of the bones.

FIG. 3 includes X-ray images of hind limbs of a mouse cultured in well A (pamidronate 1 mM) and well B (control).

FIG. 4 includes X-ray images obtained by subtracting an image of a neonatal limb culture image at 40 hours (t₀+x, wherein x=40 hours) from a neonatal limb culture image at 0 hours (t₀).

### DETAILED DESCRIPTION OF THE INVENTION

Methods of the present invention comprise imaging skeletal members in vitro. In general, the methods comprise preparing a culture comprising the skeletal member and a substance whose effect on growth of the skeletal member is to be determined and capturing images of the skeletal member at multiple selected time points to assess effects on growth of the skeletal member. The culture enables growth of the skeletal member and the images are captured without terminating growth of the skeletal member.

Culturing skeletal members is known. Traditional culture media are solid and/or liquid and are capable of enabling growth of the skeletal member and maintaining the viability of cells within the skeletal member. One suitable medium is Delbecco's Modified Eagle Medium, available commercially, and appropriately supplemented with β glycerophosphate, ascorbic acid, bovine serum albumin and antibiotics.

The skeletal member within the culture may include, for example, a limb bud, a single bone or a fully articulated skeletal member such as a neonatal foot or paw. As used herein, the term “fully articulated skeletal member” refers to a fully intact skeletal member comprising a series of interconnected bones and includes, for example, a vertebral column, a leg, an arm, a foot or a hand. The fully articulated skeletal member may be derived from any animal, including mice, rats or humans at any stage of development (e.g., neonatal, embryonic, fetal, and the like).

The fully articulated skeletal member is prepared for organ culture such that the relationships between and among the various bones of the articulated skeletal member are preserved to the degree necessary for the contemplated analysis. To obtain a fully articulated skeletal member, care is preferably taken to maintain the integrity of the relationship between bones. When removing a mouse foot, for example, the joints between the phalanges, metatarsals and tarsals are preferably not disturbed prior to culture.

The advantage to monitoring fully articulated skeletal members is that growth of each individual skeletal member is frequently influenced by neighboring members. For example, bones in the knuckles of the human hand influence development of the phalanges. By utilizing a fully articulated skeletal member, such as a substantially complete foot, the in vivo situation is more nearly replicated in vitro.

The organ culture further comprises one or more substances whose effect on growth of the skeletal member is to be determined. Such substances may include at least one cell type, drugs and combinations thereof. The at least one cell type may provide various types of growth factors or may include feeder cells.

Growth factors stimulate cellular growth and/or differentiation and include substances such as cytokines, proteins and/or steroid hormones. The growth factor may be introduced into the culture by adding purified growth factor to the culture medium or by co-culturing the skeletal member with cells that supply a growth factor. For example, bone morphogenic protein (BMP) may be provided to the culture in purified form or by co-culturing the skeletal member with cells that in turn produce and secrete BMP.

Feeder cells provide various stimuli and are useful in maintaining growth and/or development of the skeletal member over the life of the culture. Feeder cells include, but are not limited to, progenitor cells, mononuclear phagocytic cells, osteoblasts and osteoclasts. Progenitor cells are undifferentiated cells that have the capacity to differentiate into a specific cell type. Progenitor cells may be unipotent or multipotent. Feeder cells may be obtained from any source compatible with the culture under investigation, including but not limited to the same source as the skeletal member or a donor source other than the source of the skeletal member. Feeder cells may be added to the culture prior to, concurrent with or after the culture is established. Feeder cells may form a layer upon which said organ grows or they may be unattached in the culture medium.

The substance whose effect on growth is to be determined may also be a drug. Drugs useful in the methods of the invention include, for example, drugs for treating osteoporosis. A nonlimiting list of such drugs is provided in Table I.

TABLE I
Bisphosphonates

| Agent | R₁ side chain | R₂ side chain |
|---|---|---|
| Etidronate | —OH | —CH₃ |
| Clodronate | —Cl | —Cl |
| Tiludronate | —H | 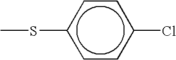 |
| Pamidronate | —OH | —CH₂—CH₂—NH₂ |
| Neridronate | —OH | —(CH₂)₅—NH₂ |
| Olpadronate | —OH | —(CH₂)₂N(CH₃)₂ |
| Alendronate | —OH | —(CH₂)₃—NH₂ |
| Ibandronate | —OH | 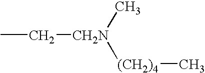 |
| Risedronate | —OH | 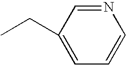 |
| Zoledronate | —OH | 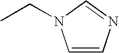 |

In addition to analyzing the effect of known drugs, drugs whose activity is unknown may also be studied. In certain embodiments, methods of the present invention are useful for screening drug candidates for drug discovery. In these embodiments, two or more cultures are prepared. A first culture, which serves as a control, enables growth of a first skeletal member in the absence of a drug candidate. A second culture enables growth of a second skeletal member and further includes one or more drug candidates whose effect on growth of the skeletal member is to be determined. Often, the skeletal members used in the first and second cultures are pairs (left and right) taken from the same animal.

Substances whose effect on growth of the skeletal member is to be determined may comprise a marker. The marker may be at least one of a biocompatible fluorescent dye, a radioactive agent and a radio-opaque agent. The marker may also be an endogenous fluorescence reporter (e.g., red fluorescent protein or green fluorescent protein (“RFP” and “GFP” respectively). The marker allows tracking of the different cell types in vitro. Biocompatible nanoparticles carrying fluorescent dyes, such as commercially available Kodak X-Sight Nanoparticles, may be employed for introducing markers into substances whose effect on growth is to be determined. As used herein, the term “biocompatible” refers to substances that do not alter the biological functions of a viable cell and/or organ and does not terminate growth of skeletal members within the culture. Feeder cells may, for example, be labeled with fluorescent dyes and tracked individually. This can be accomplished by labeling each different feeder cell with a different color of fluorescing dye or by labeling one cell type with biocompatible fluorescent dye and another with a radio-opaque material. The alternative is that each population of the cells may have different endogenous fluorescence reporter system, such as GFP or RFP.

When cells divide, the marker is advantageously carried with sister cells, allowing for tracking of families or lines of related cells. As used herein, the term “divide” includes any one of cell division, differentiation, de-differentiation, apoptosis and necrosis, either alone or in combination. Analyzing the migration patterns of different cell types helps determine various aspects of a bone feature under study. Researchers can, for example, determine what cell types contribute to the growth of individual bones and/or identify the root cause of disease states in bone based on the positioning and number of different cells.

Images of the organ culture may be captured by any method suitable to the culturing conditions, provided the imaging does not terminate growth of the skeletal member. Labeled or unlabeled skeletal members may be imaged using low-energy X-rays. Where desired, X-ray images may be enhanced by labeling cells and/or organs with radioactive and/or radio-opaque substances. Furthermore, an additional imaging mode, such as an optical mode, may be employed by labeling skeletal members or cells with at least one biocompatible dye such as a fluorescent dye. Culture images obtained by more than one method or from imaging more than one biocompatible fluorescent dye may be superimposed so as to provide a more realistic view of how various cells interact with each other and/or with the organ. In addition, imaging may be conducted over a growth cycle of the skeletal member. The term “growth cycle,” as used herein, means a period of time over which the skeletal member begins growing and then terminates growth naturally.

In addition, a method for capturing images in a multimodal fashion may be conveniently achieved by using a multi-modal system, such as the KODAK In-Vivo Multispectral Imaging System FX. Multimodal imaging involves capturing images utilizing more than one scientific technique for visualization. For example, an organ culture labeled with a biocompatible fluorescent dye and a radio-opaque agent may be visualized by both fluorescent microscopy and X-ray, the images of which may be superimposed. A system that allows for multimodal imaging is referred to herein as a “multi-modal system.”

When using a multi-modal system, the method may be quantitative and the measures may be capably enhanced using long-bone modeling to facilitate measurements of the bone density of skeletal members. The system can be used for low-energy X-ray imaging of skeletal members and limbs of neonatal animals, from a variety of sources, including but not limited to fetal, neonatal and adult animals.

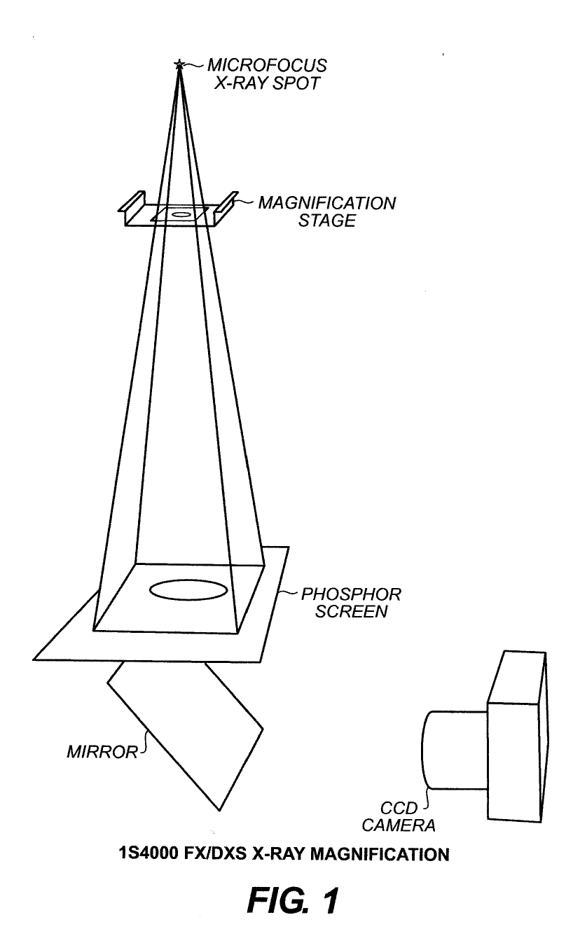

One type of multi-modal imaging system suitable for use with the method of the invention is the system as illustrated in FIG. 1. FIG. 1 shows a magnification stage used in a multi-modal imaging system useful in accordance with the present invention. The multi-modal imaging system may be of the type disclosed in co-owned, co-pending U.S. application Ser. No. 11/221,530 by Vizard et al, the disclosure of which is incorporated by reference in its entirety. As shown in FIG. 1, a low-energy X-ray beam is projected through a magnification stage to a sample or object positioned on a phosphor screen or plate that produces light in response to ionizing radiation passing through the object to be imaged. A mirror may be used to reflect the light from the phosphor plate to a CCD camera. This arrangement provides optimum spatial X-ray resolution of the object on the phosphor screen.

Images of the culture may be captured at two or more selected time points, t₀ and tₙ, where t₀ corresponds to a first time point at which imaging is conducted and tₙ corresponds to each successive time point after t₀ at which the imaging is conducted, with tₙ defined according to the equation tₙ=tₙ₋₁+x where n=an integer and x=any unit of time and where x is the same or different at each time point tₙ. The number of time points typically ranges between two and one thousand, more particularly between five and one hundred and still more particularly between ten and fifty.

Time point t₀ may be the first time point prior to exposure to an experimental condition, or it might be the first time point taken after exposure to an experimental condition. In any event, any first image or measurement taken represents a baseline to which subsequent images and/or measurement may be compared in order to determine the effect of an experimental condition.

Subsequent time points tₙ are determined according to the equation tₙ=tₙ₋₁+x. For example, capturing images at three time points, where t₀=0 and x=30 minutes, yields t₁=30 (0+30), t₂=60 (30+30) and t₃=90 (60+30). Three time points t₁, t₂ and t₃, with distinct time intervals between time points—x=30 minutes for t₁, x=45 minutes for t₂ and x=60 minutes for t₃—yields the following: t₀=0, t₁=30 (0+30), t₂=75 (30+45) and t₃=135 (75+60). The variable x may be any time measurement or fraction thereof and includes seconds, minutes, hours, days, weeks, months and years. For example, where the time points are represented in days, t₀ is day 0, and x is 0.25 for t₁, t₁=6 hours after t₀, x+0.5 for t₂, t₂=12 hours after t₁, etc.

Capturing images in this manner allows users to assess effects on growth and to examine whether and to what extent the substance whose effect on growth of the skeletal member is to be determined actually impacts growth. Assessing effects in this manner can be done without the need to terminate growth by fixing and staining as in the past. As used herein, the term “growth” refers to an increase in mass, length and/or density of a skeletal member in culture. The term “growth” also encompasses bone remodeling, including cell differentiation.

By comparing the images captured at the selected time intervals, methods of the present invention can be used to monitor growth in various ways. To monitor bone length, multiple images are compared at selected time intervals and images may be subtracted from one another. When the culture comprises radioactive labels and multi-modal imaging is employed, a pulse chase may be conducted or radio labeled ligand binding can be used to determine the number of cells in the skeletal member. In addition, users may superimpose images, including for example fluorescent images over X-ray images and thereby identify the migration of the substance whose effect on growth of the skeletal member is to be determined as a function of time. In some cases, it is advantageous to determine whether and how such migration effects skeletal growth. The positioning of certain cell types, such as osteoblasts, for example, impacts bone density. Image comparison also supports drug discovery. A comparison of images taken from a control culture against images taken from a culture comprising one or more drug candidates, enables efficient and highly effective drug screening in vitro in a manner that replicates in vivo settings. Both cultures may also include disease states, wounds or broken bones to be treated by the one or more drug candidates.

The following experimental examples are included to further illustrate embodiments of the invention. These examples are not intended to limit the invention in any manner.

### EXAMPLE 1

### Measuring Dynamic Growth of an Articulated Skeletal Member

The method of the invention may be used to monitor the dynamic growth of an articulated skeletal muscle by capturing a series of images overtime. In this example, an articulated lower portion (hind limb) of a limb of a neonatal, day-old Swiss albino mouse was removed and cultured in a well plate. The limb was scraped with a scalpel to abrade any skin and flesh sufficiently to allow an organ culture medium to reach the skeletal members. The scraped limb was superficially embedded in 1% agarose. The medium used for culture was 4 ml of minimum essential medium (Delbecco's Modified Eagle Medium), supplemented with 1 mM β glycerophosphate, ascorbic acid, 0.2% bovine serum albumin and antibiotics. The antibiotics contained 100 units/ml penicillin, and 100 μg/ml streptomycin.

The skeletal members of the limbs continued to grow within the cultures and were imaged with X-rays (Time-Lapse-3 hrs; 11 to 18 Key using a multi-modal system as previously described). Bone growth and mineral deposition could be measured, as could X-ray and column densities. Because the limb was articulated, the growth of individual skeletal members was influenced by neighboring members, just as in the case of growth in a living animal. The analytical X-ray imaging methodology according to the invention allows measurement of the growth of skeletal members of a neonatal mouse limb grown in vitro in organ culture.

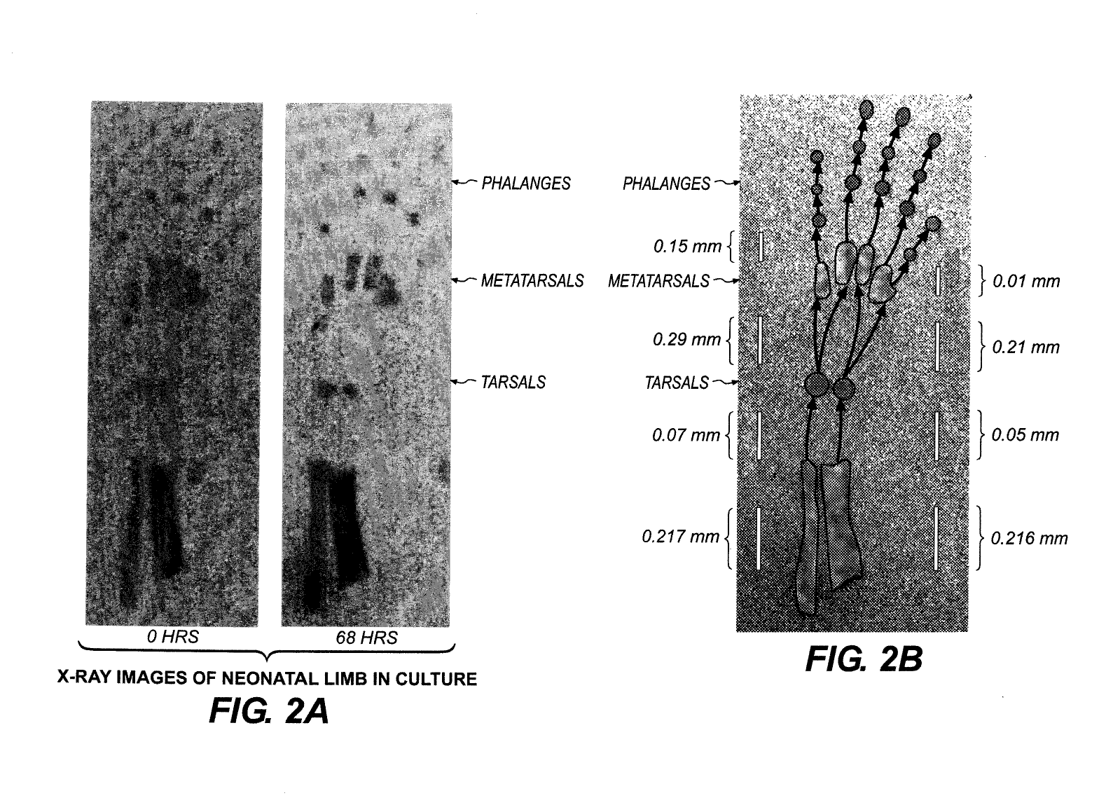

The results from the articulated skeletal member culture are provided in FIGS. 2A and 2B. The images provided in FIG. 2A depict two time spaced X-ray images of the hind limb. The left-most image shows the appearance of the tarsals, metatarsals and phalanges of the limb at zero hours (t₀) and the central image shows their appearance at 68 hours (t₁=t₀+68 hours). The right-most image shows the growth (in mm) that took place when the limb was grown for 68 hours and schematically illustrates changes in bone density, distances between the bones, and direction of bone growth.

### EXAMPLE 2

### Monitoring the Effect of Bisphosphonates on Bone Density

Pamidronates are bisphosphonates that are routinely used for prevention and treatment of osteoporosis. The action of pamidronates in early bone development is demonstrated here using the method of the invention.

Two individual limb cultures were prepared as described in Example 1. To one of the cultures, 1 mM of pamidronate was added to the culture medium. The other culture was a control with additives. An image was obtained at t₀ and another image was obtained at t₀+40 hours. The analytical X-ray imaging methodology according to the invention allows for the determination of changes in the bone density as a result of the experimental drug vs. the control.

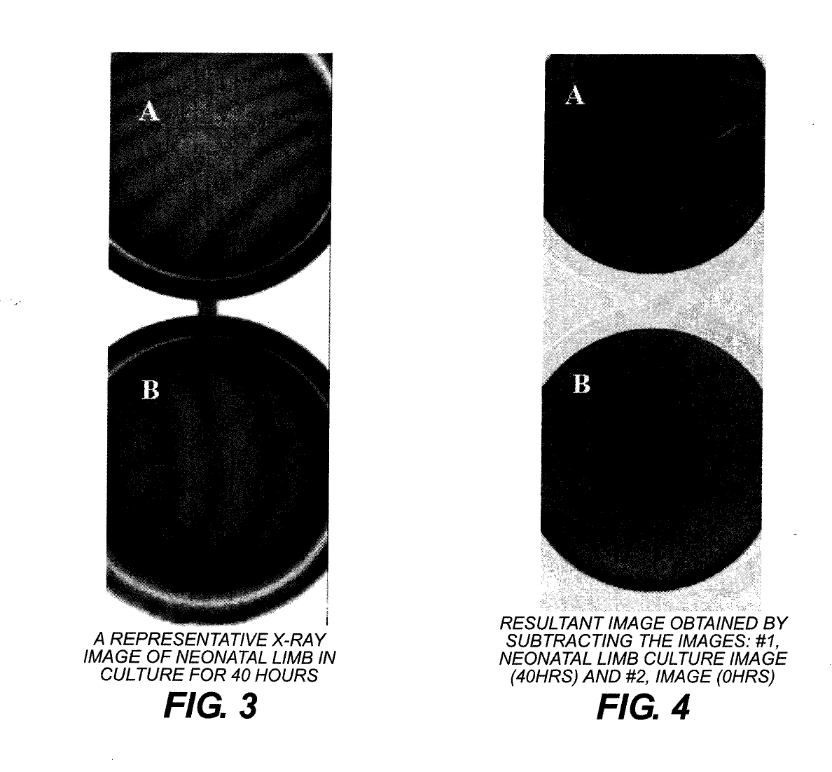

FIG. 3 shows representative X-ray images of hind limbs of a mouse cultured in well A (containing 1 mM of pamidronate) and well B (control). A representative X-ray image was captured of a neonatal limb in culture for 40 hours (15 sec exposure; f-stop 2.8; 17.18 Kev). The differences in bone density and size are observable.

FIG. 4 shows resultant X-ray images obtained by subtracting an image of a neonatal limb culture image at 40 hours from a neonatal limb culture image at 0 hours. Well A shows the difference in the images for a limb cultured with pamidronate, while well B shows the same difference in the control.

### EXAMPLE 3

### Monitoring the Replication and Differentiation of Progenitor Cells Cultured with Growth Factor (Prophetic Example)

An organ culture is prepared as described in Example 1. Alternatively, instead of starting with an articulated member from a neonatal mouse, an articulated skeletal member from an embryonic mouse may be used. A baseline image of the organ culture is obtained at t₀. Bone-related progenitor cells, either from the same mouse or a suitable donor mouse, are labeled with a fluorescent dye and added to the culture. Alternatively or additionally, the progenitor cells that are part of the initial articulated skeletal member may be labeled with the same or another fluorescent dye. Various time points (tₙ) are imaged over the life of the culture. The imaging may be obtained by fluorescent microscopy and/or X-rays. Where both imaging modes are used, the images may be superimposed on each other in order to aid in analysis.

The results from the culture described in example 3 will allow tracking of progenitor cells. When progenitor cells include endogenous reporters or are labeled with, for example, a plurality of biocompatible nanoparticles complexed with fluorescent dye, the replication of the progenitor cells may be monitored as sister cells maintain their fluorescence after division. In this way, the progenitor cells can be tracked before and after their division.

### EXAMPLE 4

### Monitoring Cells like Osteogenic Mesenchymal Cells or Macrophages and Growth Factor like BMP on Wound Healing (Prophetic Example)

An organ culture is prepared as described in Example 1. Once the culture is established, it is treated so as to create a wound on one or more bones within the fully articulated skeletal member. A first image is taken at t₀.

Mesenchymal cells or macrophages, from the organ source or from a suitable donor mouse, are co-cultured with the organ. Suitable macrophage stimulating substances are also added to the culture. Appropriate images are obtained at tₙ, over the life of the culture. By comparing between and among the images, one can determine where the macrophages traffic around and within the organ culture. In addition, through the use of X-ray analysis, one can determine the rate and extent of wound healing. Where a control culture is employed, one can determine the extent of wound healing specifically due to properly stimulated macrophages.

The invention has been described in detail with particular reference to presently preferred embodiments, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method of assessing an effect on skeletal growth comprising:
preparing a culture comprising a skeletal member and a substance whose effect on growth of the skeletal member is to be determined, the culture enabling growth of the skeletal member, and the substance whose effect on growth of the skeletal member is to be determined comprising a marker;
imaging the skeletal member at two or more selected time points, t₀ and tₙ, where t₀ corresponds to a first time point at which the multi-modal imaging is conducted and tₙ corresponds to each successive time point after t₀ at which the multi-modal imaging is conducted, with tₙ defined according to the equation tₙ=tₙ₋₁+x where n=an integer and x=a unit of time where x is the same or different at each time point tₙ, said imaging conducted without terminating growth of the skeletal member; and
identifying the positioning of the substance whose effect on growth of the skeletal member is to be determined based on the marker at the selected time points t₀ and tₙ to assess whether the positioning of the substance effects growth of the skeletal member.

2. The method of claim 1, wherein the conducting step comprises capturing two or more images of the skeletal member at the selected time points t₀ and tₙ and the method further comprises comparing the two or more images captured.

3. The method of claim 1, wherein the conducting step comprises capturing two or more images of the skeletal member at the selected time points t₀ and tₙ and the method further comprises determining the effect of the substance on growth of the skeletal member by subtracting any of the two or more images.

4. The method of claim 1, wherein the conducting step comprises capturing two or more images of the skeletal member at the selected time points t₀ and tₙ and the method further comprises superimposing any of the two or more images.

5. The method of claim 1, wherein the substance comprises a drug and the method further comprises causing a disease state in the skeletal member and determining whether the drug treats the disease state.

6. The method of claim 1, wherein the substance comprises a drug and the method further comprises injuring the skeletal member and determining whether the drug treats the injury.

7. The method of claim 1, wherein the marker exhibits at least one of fluorescence, radioactivity or radio-opacity and the identifying step comprises identifying the positioning of the marker.

8. The method of claim 1, wherein the conducting step comprises microscopic imaging.

9. A method of assessing an effect on skeletal growth comprising:
preparing a culture comprising a skeletal member and at least one cell type that effects growth of the skeletal member, the culture enabling growth of the skeletal member, and the substance whose effect on growth of the skeletal member is to be determined comprises a marker;
allowing the at least one cell type to divide into a plurality of cells
capturing images of the skeletal member at two or more selected time points, t₀ and tₙ, where t₀ corresponds to a first time point at which the imaging is conducted and tₙ corresponds to each successive time point after t₀ at which the imaging is conducted, with tₙ defined according to the equation tₙ=tₙ₋₁+x where n=an integer and x=a unit of time and x is the same or different at each time point tₙ, said imaging conducted without terminating growth of the skeletal member; and
using the images of the plurality of cells to assess an effect on growth of the skeletal member.

10. The method of claim 9, wherein the at least one cell type comprises a feeder cell or provides a growth factor.

11. The method of claim 9, wherein the plurality of cells each comprise the marker, and the using step comprising identifying the positioning of the plurality of cells based on the marker.

12. The method of claim 9, wherein the plurality of cells each comprise the marker and the using step comprising identifying a total number of the plurality of cells.

13. The method of claim 9, wherein the conducting step comprises capturing two or more images of the skeletal member at the selected time points t₀ and tₙ and the method further comprises superimposing the two or more images.

14. The method of claim 9, wherein the marker exhibits fluorescence and the conducting step comprises microscopic imaging.

15. A method of assessing an effect on skeletal growth comprising:
preparing a first culture comprising a first skeletal member, the culture enabling growth of the skeletal member;
preparing a second culture comprising a second skeletal member and one or more drug candidates whose effect on growth of the skeletal member is to be determined, the culture enabling growth of the skeletal member;
capturing images of the first and second skeletal members from each of the first and second cultures at two or more selected time points, t₀ and tₙ, where t₀ corresponds to a first time point at which the imaging is conducted and tₙ corresponds to each successive time point after t₀ at which the imaging is conducted, with tₙ defined according to the equation tₙ=tₙ₋₁+x where n=an integer and x=a unit of time and x is the same or different at each time point tₙ, said imaging conducted without terminating growth of the skeletal member; and
assessing whether the one or more drug candidates effects growth of the skeletal member by comparing the images captured at the selected time intervals t₀ and tₙ for each of the first and second cultures against each other.

16. The method of claim 15, further comprising introducing an agent into the culture that causes a disease state in the skeletal member and determining whether the drug treats the disease state.

17. The method of claim 15, further comprising subtracting the images at t₀ and tₙ.

18. The method of claim 15, wherein the at least one drug candidate further comprises a marker exhibiting at least one of fluorescence, radioactivity and radio-opacity.

19. The method of claim 18, wherein the skeletal member is fully articulated and the method further comprises repeated imaging of the skeletal member to assess effects on growth over the course of a growth cycle.

20. The method of claim 15, wherein the imaging is multi-modal.
